(19) United States
(12) Patent Application Publication
(10) Pub. No.: US 2005/0148914 A1
(43) Pub. Date: Jul. 7, 2005

(54) Adjustable range of motion limiter

(76) Inventor: Mark R. Currier, Milton, NH (US)

Correspondence Address: DAUPHIN LAW OFFICES, 9 Bartlett Street, Suite 365, Andover, MA, 01810, US

(21) Appl. No.: 10/746,850

(22) Filed: Dec. 24, 2003

(51) Int. Cl. A61F 5/00

(52) U.S. Cl. 602/5

(57) ABSTRACT

An apparatus for quietly limiting motion at a limb joint having pivotally attached members for receiving the limbs of a joint and a dual-adjustable stop mechanism assembly operatively attached to the members for restricting movement at the joint. The dual-adjustable stop mechanism assembly includes at least one removably connected adjustable range of motion limiter.

### FIELD OF THE INVENTION

The present invention relates to an ankle-foot orthosis with a dual-adjustable range of motion limiter for limiting the range of pivotal movement of the foot. Even more particularly, this invention relates to an orthotic range of motion limiter that allows for ease of manufacture of an ankle-foot orthosis and that allows the degree of plantar flexion and dorsiflexion of the foot to be adjusted and or fixed.

### BACKGROUND OF THE INVENTION

Hinged orthopedic braces having an adjustable range of pivotal movement, as disclosed by U.S. Pat. Nos. 5,022,390 (1991) and 5,328,444 (1994), both to Whiteside, are known in the art. The braces disclosed therein have adjustable stopping mechanisms to limit the range in pivotal movement of two hinged members of the brace relative to each other. The stopping mechanisms taught by the above-mentioned patents disclose an adjustable stop mechanism attached to one member of the brace that is positioned to come into contact with an abutment attached to the other member of the brace when the members are pivoted to a desired angle of restriction. To effect adjustment of this stopping mechanism, a hole must be drilled in the brace and a tool must be used.

Other ankle-foot orthotic devices such as U.S. Pat. Nos. 5,399,152 (1995) to Habermeyer et al. and 5,429,588 (1995) to Young et al. disclose ankle-foot orthoses designed to treat fractures and other injuries to the foot and ankle. Habermeyer discloses an apparatus that consists of two members pivotally connected. Each member provides surroundive fixation that is both removable and adjustable through the filling and evacuating of cushions within each member. Rear pivotal movement (plantar flexion) and front pivotal movement (dorsiflexion) are restricted by a bar with an attached abutment head contacting adjustable abutments located above and below the abutment head. One problem with this device is that the adjusting mechanism has multiple parts making it difficult and expensive to manufacture. Another problem with this device is that the parts are not easily replaceable by the wearer.

Young discloses an apparatus known in the art as a walker that consists of two members pivotally connected wherein plantar flexion and dorsiflexion are restricted between 22.5 degree of plantar flexion and 22.5 degree of dorsiflexion by adjustable screws contacting an upright side member. One problem with this device is that some wearers may require a greater range of motion than this device allows.

Still other foot and ankle devices such as U.S. Pat. Nos. 5,014,690 (1991) to Hepburn et al. and 5,144,943 (1992) to Luttrell et al. disclose dynamic splints, which apply an adjustable force inducing either plantar flexion or dorsiflexion.

U.S. Pat. No. 5,044,360 (1991) to Janke discloses a foot ankle device with two members that are pivotally attached. Dorsiflexion and plantar flexion are restricted through the use of interchangeable cams, which have differing cam surfaces that come into contact with a rotatable stop. The unique shape of each interchangeable cam determines at what point or angle in plantar flexion or dorsiflexion the rotatable stop contacts the cam surface and limits range of motion.

U.S. Pat. No. 4,919,118 (1990) to Morris discloses a short leg walker, which has a motion limiter consisting of, a shaft pivotally attached to one of the walker's members and a sliding block pivotally attached to the remaining member. Restriction of plantar flexion and dorsiflexion occurs when the sliding block engages adjustable stop members, which are located above and below the sliding block. By adjusting the positions of the stop members, the range of motion can be limited accordingly.

There remains a need to provide a strong yet lightweight comfortable ankle-foot orthosis, which can effectively limit the ankle-joint against a wide range of pivotal movement. There also remains a need to provide a strong yet lightweight comfortable ankle-foot orthosis which has few parts and which is simple and inexpensive to manufacture. Additionally, there remains a need to provide a strong yet lightweight comfortable ankle-foot orthosis, which has dual-adjustable stop members that are easy to adjust and replace, which is concealable under clothing or footwear, and which is custom fit for the wearer. Still further, there remains a need to provide a strong yet lightweight comfortable ankle-foot orthosis that can set the angle of plantar flexion of the foot and that can temporarily or permanently fix the angle of dorsiflexion, plantar flexion and pivotal movement of the foot.

### SUMMARY OF THE INVENTION

Accordingly, it is an object of the present invention to provide a comfortable ankle-foot orthosis that is strong and lightweight, which can effectively limit a wide range of plantar flexion of the ankle-joint and which is quiet in operation.

It is another object of the present invention to provide an ankle-foot orthosis that has few parts, is quiet in use and which is simple and inexpensive to manufacture.

It is yet a further object of the present invention to provide an ankle-foot orthosis that has a dual-adjustable stop mechanism that is easy to operate, which is concealable under clothing or footwear, and which is custom fit for the wearer.

It is yet a further object of the present invention to provide an ankle-foot orthosis that can set and or adjust the angle of plantar flexion of a wearer's foot and that can rigidly fix the angle of plantar flexion, dorsiflexion and pivotal movement of the wearer's foot.

Other objects will, in part, be obvious and will, in part, appear hereinafter. The invention accordingly, comprises the features of construction, combination of elements and arrangements of parts, which will be exemplified in the following detailed description and the scope of the invention will be indicated in the claims.

According to one aspect of the invention, an orthotic device for limiting motion in a limb joint comprises a first orthotic member, a second orthotic member pivotally connected to said first orthotic member, a dual-adjustable stop mechanism comprising a first range of motion limiter removably connected to and adjustable within said first orthotic member and a second range of motion limiter removably connected to and adjustable within said second orthotic member, wherein said first range of motion limiter is axially aligned to impact said second range of motion limiter.

As to another aspect of the invention, the first range of motion limiter further comprises a first notched impact stop.

As to another aspect of the invention, the second range of motion limiter further comprises a second notched impact stop.

As to another aspect of the invention, the first impact stop and or said second impact stop are fabricated from a resilient material.

As to another aspect of the invention the resilient material is polyurethane.

As to another aspect of the invention the first range of motion limiter and or said second range of motion limiter further include a cap.

As to another aspect of the invention the cap is fabricated from a resilient material.

Another aspect of the invention is directed to an adjustable range of motion limiter for use with an orthotic device comprising a first surface and a second surface, wherein said first surface is notched.

As to another aspect of the invention the second surface is concave and substantially conforms to the curvature of an orthotic device wearer's limb.

As to another aspect of the invention the adjustable range of motion limiter includes a pair of convex sides.

According to yet another aspect of the invention an orthotic device for limiting motion in a limb joint comprising a first orthotic member, a second orthotic member pivotally connected to said first orthotic member, and at least one range of motion limiter removably connected to and adjustable within said first orthotic member and or said second orthotic member comprising a first surface and a second surface, wherein said first surface is notched.

As to another aspect of the invention the second surface is concave and substantially conforms to the curvature of an orthotic device wearer's limb.

As to another aspect of the invention the at least one range of motion limiter further comprises a pair of convex sides.

As to another aspect of the invention the at least one range of motion limiter is operatively connected to both the first orthotic member and the second orthotic member.

The present invention achieves those and other objectives by providing a comfortable ankle-foot orthosis that is both strong and lightweight and that provides a single unit dual-adjustable range of motion limiter that allows for easy and inexpensive manufacture and that is quiet in use.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a perspective view of an ankle-foot orthotic device employing a dual adjustable range of motion limiter in accordance with one embodiment of the present invention;

FIG. 2 is a side elevational view of the dual adjustable range of motion limiter of FIG. 1;

FIG. 3 is an end elevational view of the dual adjustable range of motion limiter of FIG. 1;

FIG. 4 is a perspective view of an ankle-foot orthotic device employing a dual adjustable range of motion limiter in accordance with one embodiment of the present invention;

FIG. 5 is an enlarged fragmentary cross-sectional view of the dual adjustable range of motion limiter of FIG. 4 taken along line X-X;

FIG. 6 is an enlarged fragmentary cross-sectional view of the dual adjustable range of motion limiter of FIGS. 2 and 3 taken along line X-X showing the ankle-foot orthotic device and the dual adjustable range of motion limiter as separated along line B-B;

FIG. 7 is an enlarged fragmentary cross-sectional view of the adjustable range of motion limiter of FIGS. 2 and 3 taken along line B-B;

FIG. 8 is an enlarged fragmentary cross-sectional view of the adjustable range of motion limiter of FIGS. 2 and 3 taken along line B-B showing the adjustable range of motion limiter as removed from the ankle-foot orthotic device;

FIG. 9 is an enlarged fragmentary cross-sectional view of the adjustable range of motion limiter of FIGS. 2 and 3 taken along line X-X showing the ankle-foot orthotic device and the adjustable range of motion limiter as separated along line B-B and showing a pair of resulting adjustable stops as repositioned within the ankle-foot orthotic device;

FIG. 10 is an enlarged fragmentary cross-sectional view of the adjustable range of motion limiter of FIGS. 2 and 3 taken along line X-X showing the ankle-foot orthotic device and the adjustable range of motion limiter as separated along line B-B and showing a pair of resulting adjustable stops as repositioned within the ankle-foot orthotic device and further including a pair of caps on said adjustable stops.

FIG. 11 is an enlarged fragmentary cross-sectional view of the adjustable range of motion limiter of FIG. 9 showing the pair of resulting adjustable stops as removed and replaced by a single adjustable range of motion limiter;

FIG. 12 is a perspective view of the ankle foot orthotic device of FIG. 11.

### DETAILED DESCRIPTION OF THE PREFERRED EMBODIMENTS

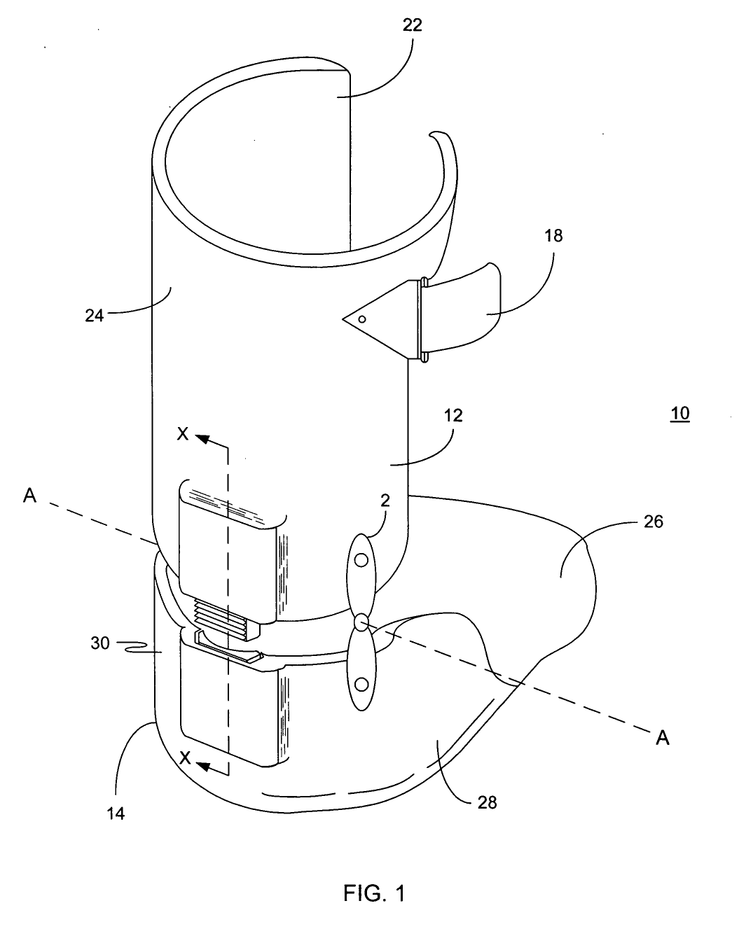

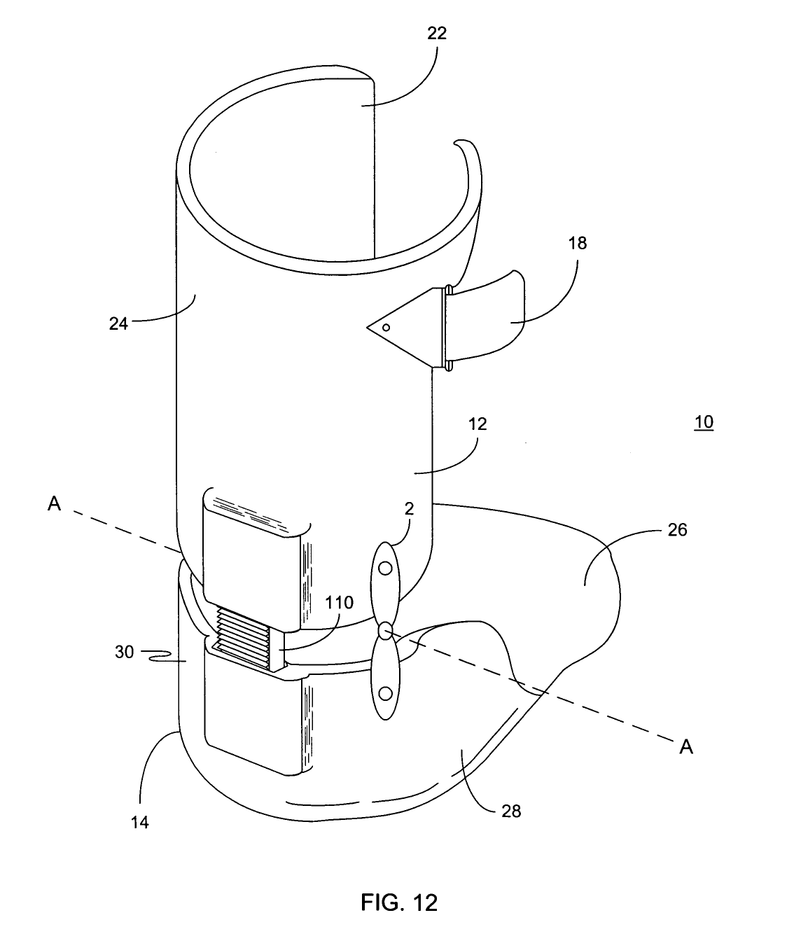

Preferred embodiments of the present invention are illustrated in FIGS. 1-12. Referring now to FIG. 1 an ankle-foot orthotic device for limiting the motion of a foot about an ankle joint is designated by numeral 10. The present description is directed primarily to an ankle-foot orthotic device; however, the method of fabrication and use of orthotic device 10 can readily be applied to other joints of the human anatomy.

Orthotic device 10 has an ankle section 12 and a foot section 14, hingedly linked to rotate about an axis A-A, by a pair of hinge mechanisms 2 and 4 (not shown). Hinge mechanisms 2 and 4 are fastened on opposite sides of orthotic device sections 12 and 14 using rivets, bolts, epoxy, or other fastening mechanism. Hinge mechanisms 2 and 4 may have moving parts like a door hinge or may be formed from a single piece of flexible material that will allow orthotic device sections 12 and 14 to pivot about axis A-A.

Ankle section 12 has an inner surface 22 for accepting a wearer's lower leg and an outer surface 24. Ankle section 12 is shown provided with an optional adjustable ankle strap 18 for securing the orthotic device to the wearer's lower leg. Adjustable ankle strap 18 may employ a VELCRO, buckle, snap or other fastening system. Foot section 14 has both an inner surface 26 for receiving the wearer's heel and foot and an outer surface 28. An optional adjustable foot strap (not shown) may be attached to foot section 14 for additional support in securing the orthotic device to the wearer's foot.

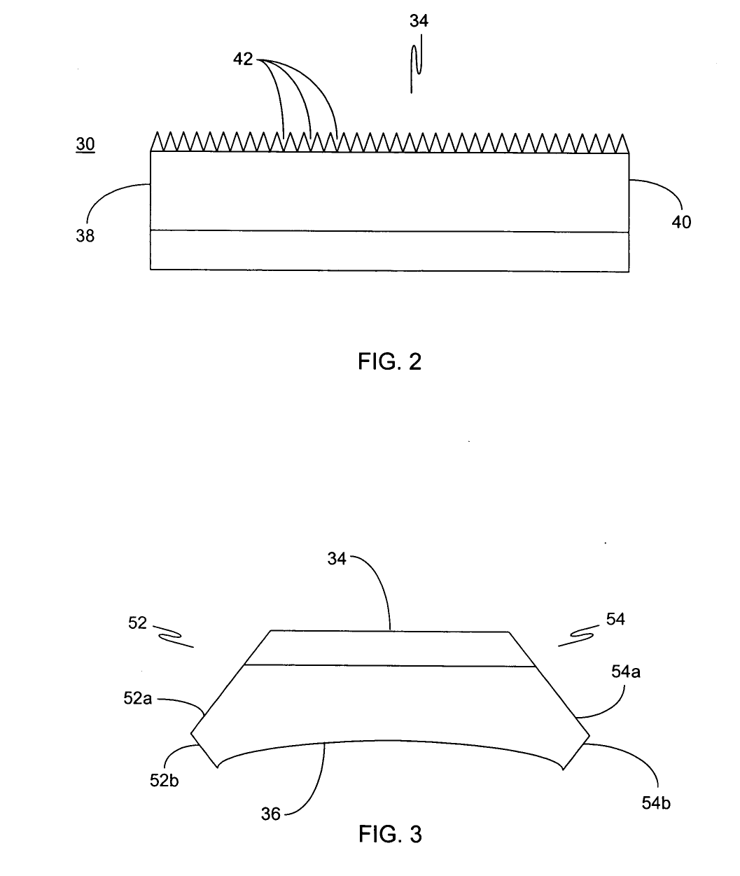

Orthotic device 10 is provided with a dual-adjustable range of motion limiter 30. The dual-adjustable range of motion limiter 30 is preferably formed of a rigid plastic or metallic material. Referring now to FIGS. 2 and 3 a side view and an end view of the dual-adjustable range of motion limiter 30 are shown, respectively. Dual-adjustable range of motion limiter 30 includes a notched upper surface 34, a curved lower surface 36, a first end 38 and a second end 40. It should be noted that the notched upper surface 34 may be internally or externally notched.

The dual-adjustable range of motion limiter 30 further includes a pair of convex sides 52, 54. In the present embodiment, the convex sides 52, 54 include upper surfaces 52a, 54a and lower surfaces 52b, 54b. Alternatively, convex sides 52, 54 may be curved or rounded as will be evident from the below description.

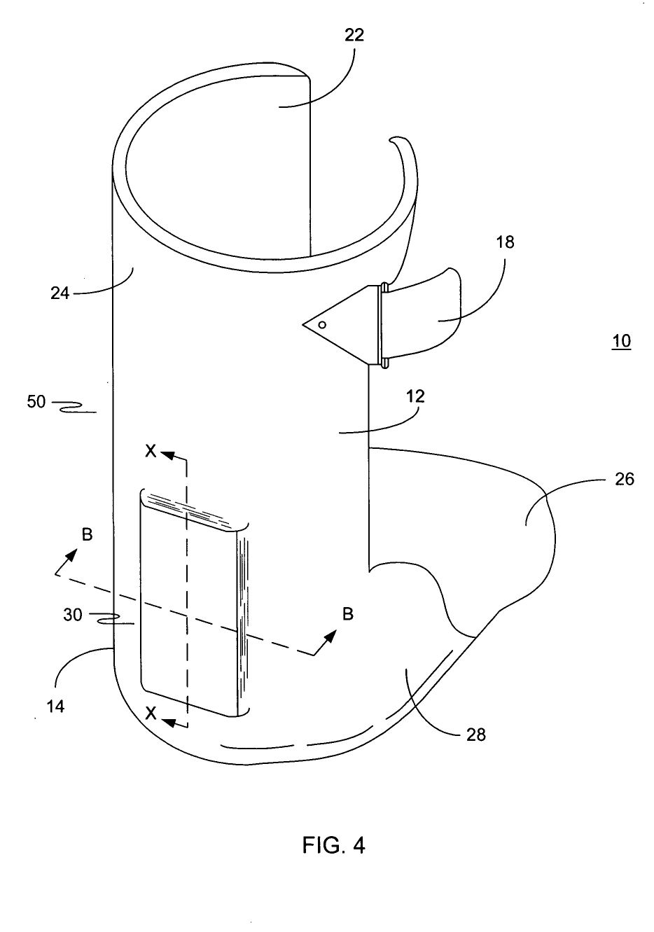

Orthotic device sections 12 and 14 are preferably formed of thermoplastic sheet material. This material is lightweight and can be shaped to intimately configure to the wearer's anatomy. To construct an ankle-foot orthotic device employing the present invention, initially, a single cast is made of the wearer's limb including the wearer's lower leg, ankle, heal and foot. From this cast a male mold (not shown) is made in configuration with the same shape of the wearer's limb. Next, the dual-adjustable range of motion limiter 30 is placed on a heal portion of the male mold as a single unit. An optional self-adhesive foam spacer (not shown) or other similar means may be employed to temporarily adhere the dual-adjustable range of motion limiter 30 to the male mold. Finally, heated sheets of thermoplastic material are superimposed over and vacuumed into intimate contact with the dual-adjustable range of motion limiter 30 and the male mold. The plastic sheets flow into a series of notches 42 of the notched upper surface 34 and around the pair of convex sides 52, 54 of the dual-adjustable range of motion limiter 30, thereby removably fixing the dual-adjustable range of motion limiter 30 within a thermoformed assemblage 50 (see FIG. 4).

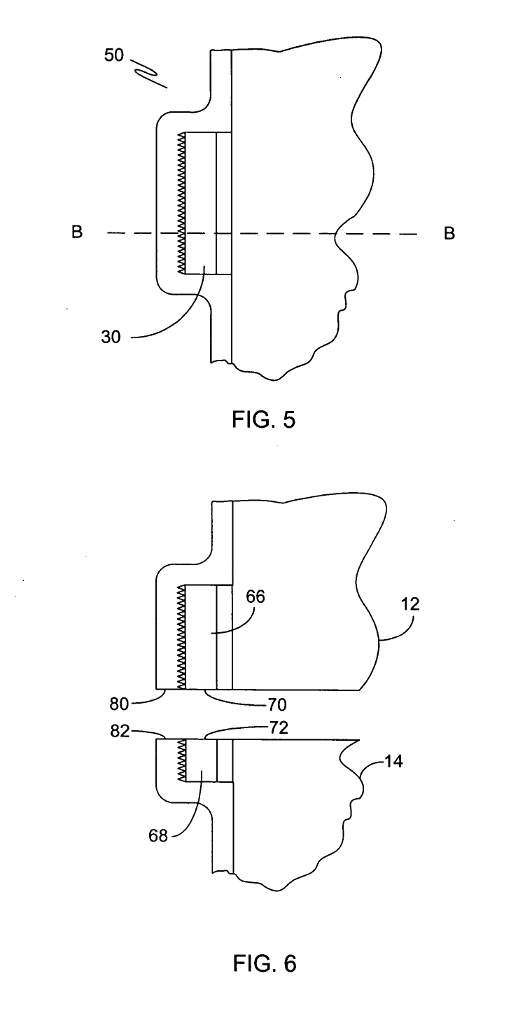

Referring now to FIGS. 5 and 6, after the thermoformed assemblage 50 is sufficiently cooled, it is sliced into two separate sections along cut line B-B, thus creating the ankle section 12 and the foot section 14. In addition to separating the thermoformed assemblage 50 into two sections, the dual-adjustable range of motion limiter 30 is similarly sectioned in two, thus creating and revealing a first impact stop 66 having an impact end 70 and a second impact stop 68 having an impact end 72. The impact stops 66, 68 are held in position by a set of layers of thermoformed plastic sheet material 80, 82.

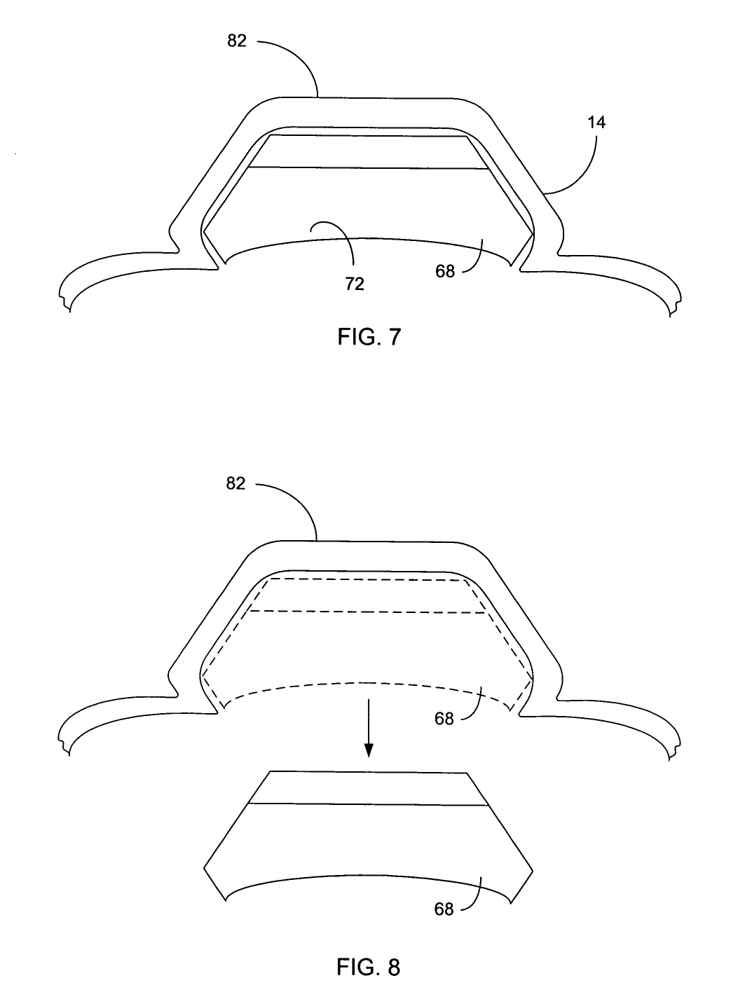

Referring now to FIGS. 7 and 8, once the impact stops 66, 68 have been exposed, either or both impact stops may be removed and or adjusted within their respective ankle and foot orthotic sections 12, 14. Preferably the layers of thermoformed plastic sheet material 80, 82 encompassing the impact stops 66, 68 and or the impact stops themselves are sufficiently pliable to allow the impact stops 66, 68 to be pried out or otherwise removed from their orthotic sections.

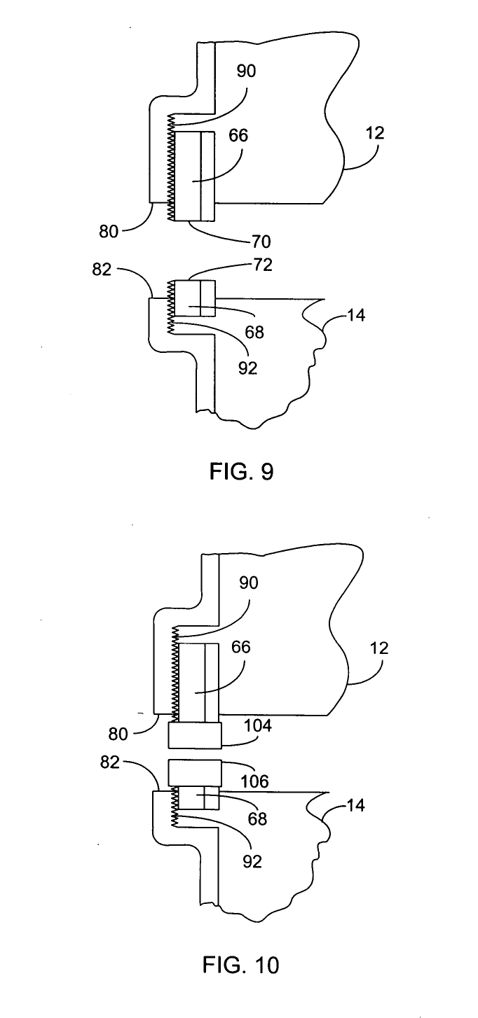

Upon removal of the impact stops 66, 68 from the ankle and foot orthotic sections 12, 14, a series of ridges 90, 92 (which are formed when the layers of thermoformed plastic sheet material 80, 82 are vacuumed into place around the series of notches 42 of the dual adjustable range of motion limiter 30) are revealed (See FIGS. 9 and 10). The uniform nature of the series of ridges 90, 92, allows impact stops 66, 68 to be repositioned in multiple positions within the ankle and foot orthotic sections 12, 14. In this and other embodiments of the present invention, the series of angled notches 42 are shown situated along upper surface 34. It is contemplated that notches or other similar indentations, protrusions or ridge configurations may be located or repositioned along other sides of the range of motion limiter 30 to achieve similar results.

In the present embodiment of the invention, repositioning either or both of the impact stops 66, 68, allows for the adjustment of the angle of plantar flexion of the wearer. As best shown in FIG. 9, the closer the impact ends 70, 72 are to each other (i.e., the further the impact stops 66, 68 protrude from the ankle and foot orthotic sections 12, 14), the smaller the angle of plantar flexion of the wearer is allowed.

In addition to repositioning the impact stops 66, 68, the wearer may replace either or both of the impact stops 66, 68 with other, alternative impact stops having similar ridge configurations. For example, longer impact stops (not shown) may be used to further limit the wearer's angle of plantar flexion. Alternatively, referring now to FIG. 10, a pair of end caps 104, 106 may be placed over the impact ends 70, 72 of the impact stops 66, 68. In the present embodiment, the end caps 104, 106 are preferably fabricated from a resilient material such as polyurethane, which provides for quiet operation of the orthotic device 10. The size of the end caps 104, 106 may be adjusted to operatively control plantar flexion. In addition, the end caps 104, 106 may be fixedly attached to impact ends 70, 72 and or used to operatively remove and or reposition the impact stops 66, 68 within the ankle and foot orthotic device sections 12 and 14.

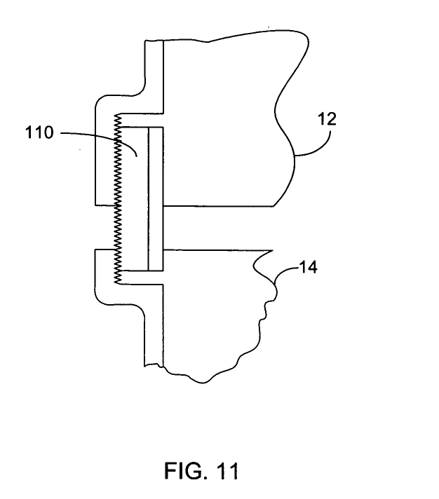

Referring now to FIG. 11, a system for fixing the angle of ankle and foot orthotic device sections 12 and 14 with respect to each other and or for completely restricting both dorsiflexion and plantar flexion is shown. In the present embodiment of the invention, the impact stops 66, 68 are removed and replaced with a single motion-limiting insert 110. The motion-limiting insert 110 prevents orthotic device sections 12 and 14 from rotating relative to one another about axis A-A, thereby restricting both dorsiflexion and plantar flexion of the wearer.

Although the preferred embodiments of the present invention have been described herein, the above descriptions are merely illustrative. Further modifications of the invention herein disclosed will occur to those skilled in the respective arts and all such modifications are deemed to be within the scope of the invention as defined by the appended claims.

1. An orthotic device for limiting motion in a limb joint comprising:
(a) a first orthotic member;
(b) a second orthotic member pivotally connected to said first orthotic member; and
(c) a dual-adjustable stop mechanism comprising:
i. a first range of motion limiter removably connected to and adjustable within said first orthotic member;
ii. a second range of motion limiter removably connected to and adjustable within said second orthotic member, wherein said first range of motion limiter is axially aligned to impact said second range of motion limiter.

2. The orthotic device as claimed in claim 1, wherein said first range of motion limiter comprises a first notched impact stop.

3. The orthotic device as claimed in claim 2, wherein said second range of motion limiter comprises a second notched impact stop.

4. The orthotic device as claimed in claim 3, wherein said first impact stop and or said second impact stop is fabricated from a resilient material.

5. The orthotic device as claimed in claim 4, wherein said resilient material is polyurethane.

6. The orthotic device as claimed in claim 1, wherein said first range of motion limiter and or said second range of motion limiter further include a cap.

7. The orthotic device as claimed in claim 6, wherein said cap is fabricated from a resilient material.

8. An adjustable range of motion limiter for use with an orthotic device comprising a first surface and a second surface, wherein said first surface is notched.

9. The adjustable range of motion limiter as claimed in claim 8, wherein said second surface is concave and substantially conforms to the curvature of an orthotic device wearer's limb.

10. The adjustable range of motion limiter as claimed in claim 8 further comprising a pair of convex sides.

11. An orthotic device for limiting motion in a limb joint comprising:
(a) a first orthotic member;
(b) a second orthotic member pivotally connected to said first orthotic member; and
(c) at least one range of motion limiter removably connected to and adjustable within said first orthotic member and or said second orthotic member comprising a first surface and a second surface, wherein said first surface is notched.

12. The orthotic device as claimed in claim 11, wherein said second surface is concave and substantially conforms to the curvature of an orthotic device wearer's limb.

13. The orthotic device as claimed in claim 11, wherein said at least one range of motion limiter further comprises a pair of convex sides.

14. The orthotic device as claimed in claim 11, wherein said at least one range of motion limiter is operatively connected to both the first orthotic member and the second orthotic member.
